(19) United States
(12) Patent Application Publication
(10) Pub. No.: US 2004/0192805 A1
(43) Pub. Date: Sep. 30, 2004

(54) Curable dental compositions

(75) Inventor: Werner Finger, Neuss (DE)

Correspondence Address: Norris, McLaughlin & Marcus P.A., 30th Floor, 220 East 42nd Street, New York, NY, 10017, US

(73) Assignee: Heraeus Kulzer GmbH & Co. KG, Hanau (DE)

(21) Appl. No.: 10/770,593

(22) Filed: Feb. 3, 2004

(30) Foreign Application Priority Data
Feb. 5, 2003 (DE) 103 04 758.1

(51) Int. Cl. A61K 6/08

(52) U.S. Cl. 523/116

(57) ABSTRACT

Curable dental compositions containing fine-particle anion exchangers exhibit excellent curing, even on dental surfaces that have been pretreated with acidic components.

The invention relates to curable dental compositions containing fine-particle anion exchangers, to a method for producing same, and to their use.

### BACKGROUND OF THE INVENTION

Curable dental compositions similar to those described in this invention find widespread application in dentistry as adhesives, adhesive dental varnishes, sealants, fixing materials, or dental filling materials, for example. Use in dental prosthetics for bonding polymeric or non-polymeric materials to metallic structural parts, for example, is also possible.

Conventional curable dental compositions contain, as an essential component. (meth)acrylate monomers, which are converted into crosslinked polymers by radically initiated polymerization and which thus bring about the solidification or curing of the dental composition. Photoinitiators, heat-activatable initiators, and redox systems are most commonly used for the initiation of polymerization.

Photoinitiators allow the formulation of one-component systems that can be cured by irradiation from intense light sources. Such a photocurable preparation for the treatment of hard dental substance is described in detail in U.S. Pat. No. 5,849,270 by way of example.

For areas of application in which curing by radiation is unfavorable or impossible, such as, for example, the attachment of crowns made of opaque material, redox polymerization can be used. In this case, two components, one containing an initiator such as dibenzoyl peroxide, and the other containing a coactivator, for example an amine such as N,N-dimethyl-p-toluidine, are intensively mixed to initiate the curing.

To achieve good adhesion of the cured dental composition to the dental substance, the dental substance can be conditioned with a liquid before the dental composition is applied. This conditioning liquid generally contains acids with a pKs less than 5 and a pH of 0.1 to 3.5. Suitable acids in this context are phosphoric acid, nitric acid, pyruvic acid, citric acid, oxalic acid, ethylene diamine tetraacetic acid, tartaric acid, and acetic acid, for example.

Instead of conditioning liquids, self-etching primers are finding increasing use, whereby the etching of the dental hard substance and the bonding to released dentin collagen are simultaneously enabled by the use of acid monomers. Such self-etching primers are easy to process, and exhibit high adhesive strength to the dental substance. In addition to acidic components such as polyacrylic acid, maleic acid, or 4-(meth)acryloxyethylphthalic acid, for example, self-curing primers contain (meth)acrylate monomers and initiators for the curing.

For certain applications, however, it has been shown that treatment with a conditioning liquid or a self-etching primer hinders the curing of subsequently applied dental compositions (Sanares A M E, Itthagarun A, King N M, Tay F R, Pashley D H. Adverse surface interactions between one-bottle light-cured adhesives and chemical-cured composites. Dental Materials 2001; 17:542-556). This hindrance is manifested by retarded or incomplete curing of the dental composition. This hindrance is particularly pronounced in the above-mentioned redox polymerization of two-component systems.

The object of the present invention, therefore, is the preparation of curable dental compositions which completely cure, even on dental surfaces treated with acidic components.

### SUMMARY OF THE INVENTION

It has been found that addition of fine-particle anion exchangers to the curable dental composition assures the complete curing of same, even when the dental surfaces have previously been treated with an acidic conditioning liquid or a self-etching primer.

The subject matter of the invention is thus curable dental compositions containing

a) Radically polymerizable monomers,

b) Radical formers and/or coinitiators,

c) Customary additives, and

d) Fine-particle anion exchangers.

The invention further relates to the use of such curable dental compositions as dental filling materials, adhesives, adhesive dental varnishes, sealants, or fixing materials, for example.

In one preferred embodiment of the present invention, the curable dental compositions are composed of two-component systems made of peroxide and amine components, the amine components containing

a) Radically polymerizable monomers,

b) Aromatic amine as coactivator,

c) Customary additives, and

d) Fine-particle anion exchangers.

### DETAILED DESCRIPTION

Radically polymerizable monomers (a) are primarily the esters of acrylic acid and methacrylic acid. Esters of (meth)acrylic acid and monovalent to pentavalent alcohols containing 2 to 30 carbon atoms are preferred. Also well suited are epoxy acrylates, such as bis-GMA of Formula

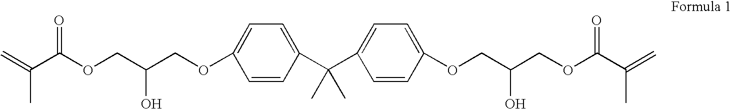

Also mentioned are urethane acrylates, which can be prepared by reacting diisocyanates and hydroxyalkyl(meth)acrylates. The following are examples of particularly well-suited urethane acrylates:

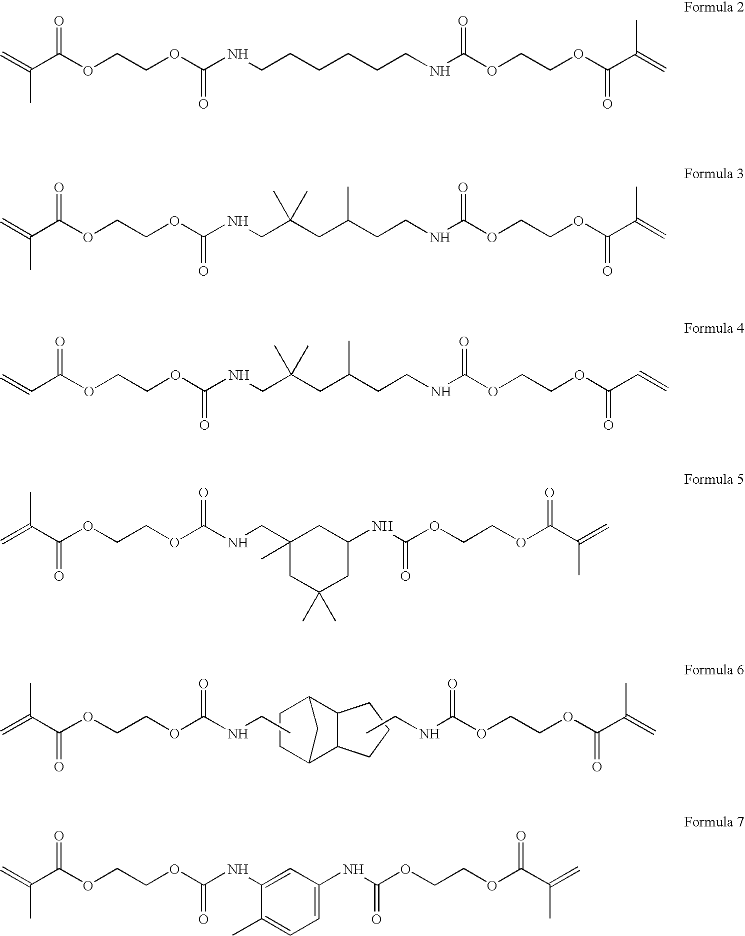

The monomers or monomer mixtures used should have a viscosity of 50 to 5000 mPa·s, preferably 100 to 2000 mPa·s (in each case at 20° C.). Higher-viscosity monomers may be mixed with low-viscosity monomers, known as reactive diluents. Examples of suitable reactive diluents include triethylene glycol dimethacrylate, tetraethylene glycol dimethacrylate, hexanediol dimethacrylate, neopentyl glycol dimethacrylate, and trimethylolpropane triacrylate. Triethylene glycol dimethacrylate is preferred. A suitable viscosity is generally achieved by the addition of 5 to 40% by weight reactive diluents (relative to the total monomers).

It has been shown to be particularly advantageous to use, at least in part, esters of acrylic acid and methacrylic acid containing free hydroxyl groups in the formulation of the dental compositions according to the invention. Hydroxyethyl methacrylate, hydroxyethyl acrylate, hydroxypropyl methacrylate, glycerin monomethacrylate, and glycerin dimethacrylate are mentioned as examples. Hydroxyethyl methacrylate is preferred. The esters of acrylic acid and methacrylic acid containing free hydroxyl groups also act as reactive diluents and reduce viscosity. The proportion of esters of acrylic acid and methacrylic acid containing free hydroxyl groups is preferably 5 to 30% by weight, relative to the total monomers.

In the present context, radical formers (b) are substances which, under the influence of light, heat, or the addition of a coinitiator, supply free radicals which are suitable for initiating a polymerization.

Light-activatable radical formers, known as photopolymerization initiators, are known from the literature. These are mono- or dicarbonyl compounds such as benzoin and derivatives thereof, in particular benzoylmethyl ether, benzil and benzil derivatives, and α-diketo derivatives, in particular those of norbornane and substituted norbornanes. Camphorquinone is preferred. The photopolymerization initiators are preferably combined with photocoinitiators such as trihexylamine, N,N-dimethylaminoethyl methacrylate, N,N, N′, N′,-tetramethyl ethylene diamine, and dialkylbarbituric acid, for example. Particularly suitable photocoinitiators are dimethylaminobenzolsulfonic acid amides according to EP 73 995. The photopolymerization initiators are used in quantities of 0.01 to 2.5% by weight, preferably 0.1 to 0.5% by weight, and the photocoinitiators in quantities of 0.02 to 4% by weight, preferably 0.2 to 1% by weight, in each case relative to the dental composition according to the invention.

Radical formers that can be activated by heat are, for example, azo compounds such as 2,2′-azobis(isobutyronitrile) and 2,2′-azobis(2-methylisobutyronitrile), and peroxy compounds such as dibenzoyl peroxide, dilauryl peroxide, bis(p-chlorobenzoyl peroxide), dicyclohexyl peroxydicarbonate, tert-butylperoxy-2-ethyl hexanoate, 2,5-bis(2-ethylhexanoylperoxy)-2,5-dimethylhexane, tert-butylperoxy benzoate, and tert-amylperoxy-2-ethylhexane. Of course, it is possible and, in many cases, advantageous to use mixtures of various radical formers, such as, for example, mixtures of radical formers having different decomposition temperatures. The radical formers are generally used in quantities of 0.05 to 2% by weight, preferably 0.1 to 0.8% by weight, relative to the dental composition.

For curing at room temperature, the dental composition according to the invention is used as a two-component system, the one component containing a peroxide as radical former and the other containing an aromatic amine as coinitiator. The composition of the peroxide and amine components can be identical with respect to their main components (radically polymerizable monomers (b) and customary additives (c)). For good stability in storage of the dental composition according to the invention, however, the fine-particle anion exchanger (d) is added to the amine component only.

Suitable radical formers for the peroxide component are primarily diacyl peroxides. Dibenzoyl peroxide, dilauryl peroxide, and bis(p-chlorobenzoyl peroxide) are mentioned by way of example. The quantity of peroxide is generally 0.05 to 2% by weight, preferably 0.1 to 0.8% by weight, relative to the peroxide component.

The amine component contains aromatic amines such as N,N-dimethyl-p-toluidine, bis-(2-hydroxyethyl)-p-toluidine, bis-(N,N-2-hydroxyethyl)-3,5-dimethylaniline, or N-methyl-N-(2-methylcarbamoyloxypropyl)-3,5-dimethylaniline, for example. The quantity of aromatic amine is 0.1 to 3% by weight, preferably 0.2 to 1% by weight, relative to the amine component.

Customary additives (c) refer to materials and substances contained in commercially available dental compositions. Included here are polymerization inhibitors such as hydroquinone monomethyl ether, resorcin, and 2,6-di-tert-butyl-4-methylphenol, for example. Premature polymerization of the dental compositions can be suppressed by the addition of polymerization inhibitors. The inhibitors are used in quantities of 50 to 5000 ppm, preferably 100 to 1000 ppm, relative to the dental composition.

As additional additives (c), the dental compositions according to the invention may contain fillers in the form of fine powder with an average particle size of 0.05 to 100 μm, preferably 0.1 to 30 μm. The fillers may be composed of silicon dioxide, quartz, aluminum oxide, glass, or glass ceramic, for example. The proportion of filler may be up to 40% by weight, preferably up to 25% by weight, relative to the dental composition.

Of course, it is possible to add common inorganic and organic pigments as additive (c) to give the dental composition according to the invention a toothlike appearance.

Suitable anion exchangers (d) according to the invention are crosslinked polymers, the amino groups, in particular dimethylamino groups, or quaternary ammonium groups as covalently bonded functional groups. The polymer base of the anion exchanger is preferably a styrene-divinylbenzene copolymer. Further particulars pertaining to structure, production, and properties of ion exchangers are described in detail in Kirk-Othmer, Encyclopedia of Chemical Technology,

Fourth Edition, Volume 14, p. 737 ff, for example. Preferred are the strongly basic anion exchangers, i.e., anion exchangers containing trimethylammonium groups orhydroxyethyldimethylammonium groups as functional groups. Gel-like as well as macroporous anion exchangers may also be used. The strongly basic ion exchangers are preferably used in the OH form.

The particle size of the anion exchangers used for the present invention is 0.1 to 100 μm, preferably 0.5 to 50 μm, particularly preferably 1 to 25 μm. These particle sizes are suitably produced by grinding coarser commercially available anion exchangers. Ball mills and bead mills have proven to be particularly useful in this regard. The use of liquid nitrogen for cooling the grinding stock is not absolutely necessary, but it simplifies the comminution, especially when particularly small particle sizes are sought.

Anion exchangers are typically marketed as water-containing gels with a water content of 35 to 70%. For the use according to the invention, the water content is reduced to less than 10%, preferably less than 2%, by drying. The drying can take place in, for example, a drying oven at temperatures of 60 to 100° C. and pressures of 0.05 to 1.0 bar. It is practical to perform the drying before the comminution. Correspondingly, the invention further relates to a method for producing curable dental compositions, in which, for a commercially available anion exchanger, the water content is reduced to less than 10% by drying, and the anion exchanger is comminuted and subsequently added to components

a) Radically polymerizable monomers,

b) Radical formers and/or coinitiators, and

c) Customary additives as constituent d).

The weight percentage of the anion exchanger in the curable dental composition is generally 0.5 to 50%, preferably 4 to 20%.

Surprisingly, it was found that, after curing of the dental composition, the fine-particle anion exchangers are firmly and durably anchored in the material formed, and no impairment of the mechanical properties of the material can be observed.

The following examples explain the invention without limiting it.

### EXAMPLES

The dental compositions according to the invention were produced by intensive mixing of the components listed in the following Examples 1 through 4.

### Example 1

BPO component (in portions by weight)

| | |
|---|---|
| 80 | Urethane dimethacrylate according to Formula 3 |
| 10 | 2-Hydroxyethyl methacrylate |
| 10 | Triethylene glycol dimethacrylate |
| 0.3 | Benzoyl peroxide |

Amine component (in portions by weight)

| | |
|---|---|
| 80 | Urethane dimethacrylate according to Formula 3 |
| 10 | 2-Hydroxyethyl methacrylate |
| 10 | Triethylene glycol dimethacrylate |
| 0.5 | Dimethyl-p-toluidine |

### Example 2

BPO component (in portions by weight)

| | |
|---|---|
| 80 | Urethane dimethacrylate according to Formula 3 |
| 10 | 2-Hydroxyethyl methacrylate |
| 10 | Triethylene glycol dimethacrylate |
| 0.3 | Benzoyl peroxide |

Amine component (in portions by weight)

| | |
|---|---|
| 80 | Urethane dimethacrylate according to Formula 3 |
| 10 | 2-Hydroxyethyl methacrylate |
| 10 | Triethylene glycol dimethacrylate |
| 8 | Strongly basic anionic ion exchanger in the OH form |
| | with a water content of 0.2% by weight and an |
| | average particle size of approximately 10 μm |
| | and a maximum particle size of approximately 100 μm. |
| 0.5 | Dimethyl-p-toluidine |

### Example 3

BPO component (in portions by weight)

| | |
|---|---|
| 80 | Urethane dimethacrylate according to Formula 3 |
| 10 | 2-Hydroxyethyl methacrylate |
| 10 | Triethylene glycol dimethacrylate |
| 0.3 | Benzoyl peroxide |

Amine component (in portions by weight)

| | |
|---|---|
| 80 | Urethane dimethacrylate according to Formula 3 |
| 10 | 2-Hydroxyethyl methacrylate |
| 10 | Triethylene glycol dimethacrylate |
| 16 | Anionic ion exchanger as described in Example 2 |
| 0.5 | Dimethyl-p-toluidine |

### Example 4

BPO component (in portions by weight)

| | |
|---|---|
| 80 | Urethane dimethacrylate according to Formula 3 |
| 10 | 2-Hydroxyethyl methacrylate |
| 10 | Triethylene glycol dimethacrylate |
| 0.3 | Benzoyl peroxide |

Amine component (in portions by weight)

| | |
|---|---|
| 80 | Urethane dimethacrylate according to Formula 3 |
| 10 | 2-Hydroxyethyl methacrylate |
| 10 | Triethylene glycol dimethacrylate |
| 32 | Anionic ion exchanger as described in Example 2 |
| 0.5 | Dimethyl-p-toluidine |

### Example 5

### Application Test, Bonding Strength to Enamel and Dentin

The effectiveness and suitability of the dental compositions as bonding components were tested in combination with a self-etching primer adhesive by determining the shear bonding strength on enamel and dentin. Extracted human molars kept in 1% chloramine solution for a maximum of six months following extraction were carefully cleaned with deionized water and dried with compressed air, and embedded in cylindrical rubber molds (diameter: 25 mm; height; 15 mm) using epoxide resin (Lekutherm X20, T1 curing agent) in such a way that an intact proximal surface rested on the base of the rubber mold. After 24 hours curing time at room temperature (23° C.), the embedded samples were released from the molds, and were wet ground from the base side on SiC paper of 180, 240, 320, 400 and finally 600 grit until a sufficiently large peripheral enamel or dentin surface was exposed for bonding a composite cylinder having a diameter of 3.5 mm.

After washing with deionized water and drying in a compressed air stream, the dental surfaces thus exposed received an application of the commercially available self-etching primer adhesive iBond Gluma inside (pH 2.2) from Heraeus Kulzer GmbH & Co. KG, using a saturated brush, in three coats directly following one another, which was left on the treated surface undisturbed for 30 seconds. The layer was then carefully freed of solvent (acetone and water) for 5 to 10 seconds in a gentle compressed air stream. In a first test series, the adhesive was not light-activated. In a second test series, iBond was activated for 30 seconds using the Translux CL polymerization light (Heraeus Kulzer; 600 mW/cm² power).

The pretreated samples were clamped in a clamping device under a divisible Teflon mold (diameter 3.5 mm, height 2.0 mm). As redox-activated composite, the core buildup material Core Paste (Den-Mat, Santa Maria, Calif., USA) was mixed from equal volume portions of base paste and catalyst paste according to the manufacturer's instructions, taken up in an application syringe, introduced into the Teflon mold, covered with an O₂-impermeable strip, and left for 15 minutes at room temperature. Dental surfaces, pretreated with iBond, on which the Core Paste material was directly applied without an intermediate layer were used as reference samples. The samples were then released from the mold and kept in warm water at 37° C. for 24 hours.

For testing the formulations according to the invention (Examples 1 through 4), the BPO- and amine-containing components were mixed in equal volume portions and applied in one layer, using a saturated brush, to the dental surface pretreated with iBond, and were left undisturbed for 60 seconds before the monomer coat was blown with a gentle air stream to give a uniformly thin layer. After the samples thus pretreated were clamped under a Teflon mold, as described above the core buildup material Core Paste was applied to the samples, which were kept in water at 37° C. for 24 hours.

The samples were clamped in a universal testing machine and, with the use of a pressure piston, load was applied to the plastic cylinder, parallel to and at a distance of approximately 0.2 mm from the dental surface, at an advance rate of 1 mm/minute until rupture.

The shear bonding strength is calculated from the quotient of the rupture force and the bonding surface of the cylinder, and is expressed in MPa.

Each sheared-off sample was examined on the tooth side for fracture morphology in a stereomicroscope at 20× magnification, and was classified as cohesive failure in the tooth or plastic, as adhesive failure at the interface, or as a mixed fracture (adhesive-cohesive).

In further studies the influence of shortened retention times (10 or 30 seconds) for the inventive formulations on the bonding strength was tested.

The results are compiled in the following Tables 1 through 12:

TABLE 1
Shear bonding strength on dentin
(no light activation of iBond)

Shear bonding
strength (MPa)

| Preparation | Average | Standard | Significant |
|---|---|---|---|
| according | value (MPa) | deviation | differences |
| to Example No. | n = 6 | ±(σₙ₋₁) | (p < 0.05) |
| Reference | 0.0 | — | a |
| 1 | 8.5 | 1.5 | b |
| 2 | 19.9 | 4.1 | c |
| 3 | 19.7 | 7.4 | c |
| 4 | 22.8 | 8.0 | c |

Analysis of variance: p < 0.0001. Values denoted by the same letters indicate no significant difference between values (Duncan ranking: p < 0.05).

No bonding to dentin was achieved for any of the reference samples without a bond intermediate layer. All samples spontaneously came loose when removed from the Teflon mold. Preparation 1, which was not provided with ion exchanger, exhibited a bonding strength that was significantly lower than that of the samples coated with preparations 2 through 4, while no statistical difference was detected in the effectiveness of the preparations having different ion exchanger concentrations. The failure of the reference samples can presumably be explained in part by the effect of the acidic monomer components of iBond, which deactivated the amine component of the composite. At the same time, it can be assumed that, due to the O₂ inhibition of the iBond polymerization occurring by diffusion of oxygen on the free side as well as continuously on the dentin side, and due to the slow redox polymerization of the composite, hardening at the interface is prevented. If a layer of preparation 1, which contains no ion exchanger, is applied, polymerization takes place, in contrast, at the interface with the dentin. Preparation 1 bonds within three to four minutes after the components are mixed together; i.e., the diffusion time for the acidic monomer is relatively short, and complete deactivation of the amine component in the experimental preparation is prevented. Preparations containing ion exchanger in the tested concentrations of 8, 16, or 32% in the amine solution are equally effective components for achieving high bonding strength to dentin. The effectiveness may be explained only by the inventive protective function of the amine component as the result of the basic ion exchanger in the redox-activated preparations, since the bonding time for the mixtures does not differ from that for preparation 1.

TABLE 2
Shear bonding strength to dentin
(30-second light activation of iBond)

Shear bonding
strength (MPa)

| Preparation | Average | Standard | Significant |
|---|---|---|---|
| according | value (MPa) | deviation | differences |
| to Example No. | n = 6 | ±(σₙ₋₁) | (p < 0.05) |
| Reference | 0.0 | — | a |
| 1 | 5.2 | 4.6 | b |
| 2 | 13.8 | 3.4 | c |
| 3 | 17.7 | 5.1 | c |
| 4 | 15.8 | 1.4 | c |

Analysis of variance: p < 0.0001. Values denoted by the same letters indicate no significant difference between values (Duncan ranking: p < 0.05).

For the preparations according to Examples 1 through 4, light activation of iBond resulted in bonding strength to dentin that was lower than for non-activated iBond layers. However, even with this procedure the effectiveness of the samples containing ion exchanger was significantly better than that for mixture 1 not subjected to load, and the reference.

TABLE 3
Shear bonding strength to dentin as a function of retention
time for the preparation according to Example 2
(no light activation of iBond)

| | Shear bonding strength (MPa) | | |
|---|---|---|---|
| | Average value | Standard | |
| Retention time | (MPa) | deviation | Significant differences |
| (seconds) | n = 6 | ±(σₙ₋₁) | (p < 0.05) |
| 10 | 17.0 | 3.5 | a |
| 30 | 21.2 | 5.0 | a |
| 60 | 19.9 | 4.1 | a |

Analysis of variance: p = 0.2462. Values denoted by the same letters indicate no significant difference between values (Duncan ranking: p < 0.05).

The tested shorter retention times (10 and 30 seconds) for the preparation according to Example 2 on dentin treated with iBond were not significantly different from a retention time of one minute.

TABLE 4
Shear bonding strength to dentin as a function of retention
time for the preparation according to Example 2
(30-second light activation of iBond)

| | Shear bonding strength (MPa) | | |
|---|---|---|---|
| | Average value | Standard | |
| Retention time | (MPa) | deviation | Significant differences |
| (seconds) | n = 6 | ±(σₙ₋₁) | (p < 0.05) |
| 10 | 14.2 | 3.0 | a |
| 30 | 17.7 | 4.4 | a |
| 60 | 13.8 | 3.4 | a |

Analysis of variance: p = 0.1725. Values denoted by the same letters indicate no significant difference between values (Duncan ranking: p > 0.05).

The tested shorter retention times (10 and 30 seconds) for the preparation according to Example 2 on dentin which was treated with iBond and light activated were not significantly different from a retention time of one minute. The average bonding strength, on the other hand, was lower than in the group without light activation of iBond (Table 3).

TABLE 5
Shear bonding strength to enamel
(no light activation of iBond)

| | Shear bonding strength (MPa) | | |
|---|---|---|---|
| Preparation | Average | | |
| according to | value (MPa) | Standard deviation | Significant differences |
| Example No. | n = 6 | ±(σₙ₋₁) | (p < 0.05) |
| Reference | 21.5 | 6.5 | a |
| 1 | 31.8 | 5.5 | b |
| 2 | 27.4 | 5.3 | a, b |
| 3 | 25.7 | 3.3 | a, b |
| 4 | 28.8 | 2.5 | b |

Analysis of variance: p = 0.017. Values denoted by the same letters indicate no significant difference between values (Duncan ranking: p < 0.05).

The reference samples without bond intermediate layers exhibited a lower bonding strength than did the samples coated with preparations 1 through 4. Hypothetically, the surprisingly high bonding strength of the reference samples may be explained by the fact that the thickness of the iBond layer is only extraordinarily low because of the very small etching depth on the enamel, the thorough blowing after application, and displacement by the comparatively high viscosity of the composite paste. The very low volume of acidic monomer, composed of iBond, acting on the amine component of the composite during diffusion is presumably too small to prevent polymerization along the interface. At the same time, the diffusion of the composite monomers into the nonpolymerized (light-activatable) iBond layer probably contributes to polymerization of the adhesive due to radical transfer. According to the statistical analysis, preparations 1 through 4 are considered to be equally effective.

The bonding strengths do not provide any indication that addition of the ion exchanger in the selected concentrations has an influence on the effectiveness on enamel.

TABLE 6
Shear bonding strength to enamel
(30-second light activation of iBond)

| | Shear bonding strength (MPa) | | |
|---|---|---|---|
| Preparation | Average | | |
| according to | value (MPa) | Standard deviation | Significant differences |
| Example No. | n = 6 | ±(σₙ₋₁) | (p < 0.05) |
| Reference | 25.0 | 4.7 | a |
| 1 | 27.6 | 4.1 | a |
| 2 | 28.7 | 3.9 | a |
| 3 | 29.5 | 3.5 | a |
| 4 | 27.1 | 5.1 | a |

Analysis of variance: p = 0.017. Values denoted by the same letters indicate no significant difference between values (Duncan ranking: p < 0.05).

All samples, including the reference, exhibited comparatively high bonding strengths to enamel. The slightly higher strength of the reference compared to the group not previously illuminated can probably be attributed to the prepolymerization of iBond, after which only the volume of the iBond inhibition layer, very much smaller by this time, is available as an acid supplier and thus as the agent compromising the amine in the redox components.

TABLE 7
Shear bonding strength to enamel as a function of retention
time for the preparation according to Example 2
(no light activation of iBond)

| | Shear bonding strength (MPa) | | |
|---|---|---|---|
| | Average value | Standard | |
| Retention time | (MPa) | deviation | Significant differences |
| (seconds) | n = 6 | ±(σₙ₋₁) | (p < 0.05) |
| 10 | 29.4 | 4.2 | b |
| 30 | 22.2 | 5.8 | a |
| 60 | 27.4 | 5.3 | a, b |

Analysis of variance: p = 0.0680. Values denoted by the same letters indicate no significant difference between values (Duncan ranking: p < 0.05).

The tested retention times for preparation 2 showed no difference in bonding strength to enamel, Therefore, ten seconds was considered to be sufficient.

TABLE 8
Shear bonding strength to enamel as a function of retention
time for the preparation according to Example 2
(30-second light activation of iBond)

| | Shear bonding strength (MPa) | | |
|---|---|---|---|
| | Average value | Standard | |
| Retention time | (MPa) | deviation | Significant differences |
| (seconds) | n = 6 | ±(σₙ₋₁) | (p < 0.05) |
| 10 | 26.0 | 3.5 | a |
| 30 | 26.8 | 4.4 | a |
| 60 | 28.7 | 3.9 | a |

Analysis of variance: p = 0.4958. Values denoted by the same letters indicate no significant difference between values (Duncan ranking: p < 0.05).

The tested retention times for preparation 2, even after light activation of iBond, showed no difference in bonding strength to enamel.

In the stereomicroscopic evaluation of the sheared-off samples (Tables 9 through 12), the reference samples exhibited adhesive failure on dentin only. Almost all the other samples, for both enamel and dentin, exhibited cohesive fractures. Of the 48 dentin samples provided with preparations 1 through 4, the fracture was located in the resin in 42 cases, in the dentin in two cases, and at the interface in four cases. Of the 60 total enamel samples, 24 cases showed resin fractures and 36 cases showed enamel fractures. Cohesive-adhesive mixed fractures were not encountered. The occurrence of cohesive failures in the dental hard substance or in the grafted resin clearly shows that the bonding at the interface is stronger than that in the substrate situated above or below.

TABLE 9
Fracture morphology (dentin)
(no light activation of iBond)

| Preparation according | Tooth | Resin | Interface |
|---|---|---|---|
| to Example No. | (cohesive) | (cohesive) | (adhesive) |
| Reference | 0 | 0 | 6 |
| 1 | 0 | 4 | 2 |
| 2 | 0 | 5 | 1 |
| 3 | 0 | 5 | 1 |
| 4 | 1 | 5 | 0 |

TABLE 10
Fracture morphology (dentin)
(30-second light activation of iBond)

| Preparation according | Tooth | Resin | Interface |
|---|---|---|---|
| to Example No. | (cohesive) | (cohesive) | (adhesive) |
| Reference | 0 | 0 | 6 |
| 1 | 0 | 6 | 0 |
| 2 | 1 | 5 | 0 |
| 3 | 0 | 6 | 0 |
| 4 | 0 | 6 | 0 |

TABLE 11
Fracture morphology (enamel)
(no light activation of iBond)

| Preparation according | Tooth | Resin | Interface |
|---|---|---|---|
| to Example No. | (cohesive) | (cohesive) | (adhesive) |
| Reference | 2 | 4 | 0 |
| 1 | 5 | 1 | 0 |
| 2 | 5 | 1 | 0 |
| 3 | 4 | 2 | 0 |
| 4 | 3 | 3 | 0 |

TABLE 12
Fracture morphology (enamel)
(30-second light activation of iBond

| Preparation according | Tooth | Resin | Interface |
|---|---|---|---|
| to Example No. | (cohesive) | (cohesive) | (adhesive) |
| Reference | 2 | 4 | 0 |
| 1 | 4 | 2 | 0 |
| 2 | 4 | 2 | 0 |
| 3 | 4 | 2 | 0 |
| 4 | 3 | 3 | 0 |

I claim:

1. Curable dental compositions comprised of a two-component system made of peroxide and amine components for curing dental surfaces treated with acidic components, the amine components comprising
a) Radically polymerizable monomers,
b) Radical formers, or coinitiators, or both,
c) Customary additives, and
d) Strongly basic anion exchangers in the OH form, having a particle size of from 0.1 to 100 μm.

2. Curable dental composition according to claim 1, wherein the weight percentage of the anion exchanger is 0.5 to 50%, based on the weight of the dental composition.

3. Method for producing the curable dental composition of claim 1, which comprises reducing the water content of a strongly basic anion exchanger in the OH form to less than 10% by weight, by drying, comminuting the anion exchanger and subsequently added the comminuted anion exchanger to components a), b) and c) of claim 1.

4. Method according to claim 3, wherein the water content is reduced to less than 2% by weight.

5. A dental filling material, adhesive, adhesive dental varnishe, sealants, or fixing material comprising the curable dental composition of claim 1.
